(19) United States
(12) Patent Application Publication
(10) Pub. No.: US 2006/0228324 A1
(43) Pub. Date: Oct. 12, 2006

(54) Decorative printing on polymeric gel air freshener and methods

(76) Inventor: Yuh-Jye Uang, St. Louis, MO (US)

Correspondence Address: Paul M. Denk;Ste. 170, 763 S. New Ballas Road, St. Louis, MO, 63141, US

(21) Appl. No.: 11/401,817

(22) Filed: Apr. 11, 2006

Related U.S. Application Data
(60) Provisional application No. 60/669,989, filed on Apr. 11, 2005.

(51) Int. Cl. A61L 9/00

(52) U.S. Cl. 424/76.2

(57) ABSTRACT

The present invention relates to provide decorative printing on a surface of polymeric gel materials. The printing methods can be pad printing, or screen-printing and heat transfer printing. The polymeric gel material is also capable of releasing a fragrance. The gel air freshener can be hang on the rear mirror inside the vehicles and also can stick any smooth surface such as window glass, ceramic tile, polished plastic and metals. The polymeric gel air freshener of this invention contains thermoplastic rubber copolymers, hydrocarbon oil, fragrances and functional additives. The invention further provides methods of making articles manufacture that the gel air freshener includes ornament beads and a string or elastic string through a loop to suspend the air freshener. The ornamental beads can be a variety of shapes, each having through-holes for stringing on the tie string. The materials selected for the ornament bead can be any polymers, which can be optionally impregnated with fragrance.

### CROSS REFERENCE TO REALTED APPLICATION

This non-provisional patent application claims priority to the provisional patent application having Ser. No. 60/669,989, which was filed on Apr. 11, 2005.

### FIELD OF THE INVENTION

The present invention relates to provide decorative printing on a surface of polymeric gel materials. The printing methods can be pad printing, or screen-printing and heat transfer printing. The polymeric gel material is also capable of releasing a fragrance. The gel air freshener can be hang on the rear mirror inside the vehicles and also can stick any smooth surface such as window glass, ceramic tile, polished plastic and metals.

The polymeric gel air freshener of this invention contains thermoplastic rubber copolymers, hydrocarbon oil, and functional additives. The thermoplastic rubbers preferably derived from triblock styrenic rubber copolymer such as hydrogenated poly-isoprene/butadiene (SEEPS) polymer. The hydrocarbon oil is mineral oil.

### BACKGROUND OF THE INVENTION

A variety of hydrocarbon gel products are on the market. There are used mostly for toys, novelty, gifts, window cling, and decorative ornaments. The customers are particularly attractive by the hydrocarbon gel products due to its features of soft, clear, stretchable, and removable. However, the hydrocarbon gel product does not allow easily for any additional visuals, be it by print. Much of this may be related to the nature of the gel product in surface structure containing mineral oil. It would be quite desirable to provide a way of printed detail graphic into hydrocarbon gel product, without reducing the amount of surface area that contributes to traction of these gel products by addition of an ink printed surface areas. Additionally, the careful selection of the composition of gel products has related the good adhesion between ink film and surface of the gel products.

U.S. Pat. No. 4,369,284 describes a transparent gel prepared from triblock copolymers and oils useful as molded products. The triblock copolymers used therein receive specific styrene end blocks to ethylene and butylene center blocks. The end block to center block ratio is given as being between 31:69 to 40:60.

U.S. Pat. No. 5,618,882 discloses the gel composition comprises styrene-(ethylene/propylene) styrene(SEPS) block copolymer having Mw of at least 180,000 and polystyrene content of 25-45 weight percent. The SEPS gel also tends to have higher tack than known SEBS gel.

U.S. Pat. No. 5,871,765 describes the controlled release polymer gel air care products, such as an air freshener, and the like. The air care product employs a hydrocarbon gel that contains block copolymer blends, the copolymers being preferably derived from styrene-rubber block units. The triblock, however, is made from Kraton G1650 copolymer comprises a styrene-ethylene-butylene-styrene structure.

U.S. Pat. No. 5,884,639 discloses a novel crystal gels and articles are formed from one or more of linear SEBS or radial (SEB)n triblock copolymers having a selected crystalline midblock segment and high levels of a plasticizer.

U.S. Pat. No. 6,309,715 discloses a fragrant article that delivers fragrance over a period of time by an article comprising a polymer matrix, a fragrance and a decorative object.

U.S. Pat. No. 6,500,218 discloses a novel transparent stiff gel candles comprising a hydrocarbon oil, one or more hydrogenated triblock copolymer of thermoplastic rubber. The used triblock copolymer is Septon 4033, are available as a hydrogenated poly isoprene/butadiene (SEEPS) polymer.

The current methods for applying images to thermoplastic rubber gel products include hand painting and spry. Since the composition of thermoplastic rubber gel contain a lot of mineral oil, which is up to 80%, ink film is very difficult to stay on the surface of the gel products. The present invention has developed the flexible printing ink and decorative printing image can be accomplished by using pad printing ink, or screen printing ink then heat release transfer printing. The top coat is necessary to protect the applied image.

### SUMMARY OF THE INVENTION

The present invention is to provide hydrocarbon gel air freshener, the surface of said air freshener possessing adhesive properties. By providing a positive adhesive, the gel air freshener itself will adhere to surfaces. It is object of this invention to provide a gelled hydrocarbon composition, suitable for use as an air freshener, comprising a hydrocarbon oil gelled a blend of triblock styrenic rubber copolymer, and fragrance oil.

Another object of the invention is to provide stiff, flexible, bendable and removable gel air freshener through careful selection of different molecular weight of the thermoplastic rubber copolymer and different viscosity of mineral oils. A further object of this invention is to print ink image onto the surface of the gel by pad printing, screen printing and then heat transfer methods. Both gel air freshener and printing ink can further comprise luminescent pigment, fluorescent pigment, or thermochromic pigment.

The invention further provides methods of making articles manufacture that the gel air freshener includes ornament beads and a string or elastic string through a loop to suspend the air freshener. The ornamental beads can be a variety of shapes, each having a through-hole for stringing on the tie string. The materials selected for the ornament bead can be any polymers, which can be optionally impregnated with fragrance.

### DETAILED DESCRIPTION OF THE INVENTION

The present invention is directed a decorative printing on a polymeric gel air freshener. The polymeric gel air freshener of this invention contains thermoplastic rubber copolymers, hydrocarbon oil, and functional additives. The gel air freshener includes a string or elastic string through a loop to suspend the air freshener. The printing methods include pad printing, or screen printing and heat transfer printing. The method of decorative printing is depending the size of the gel air freshener. The screen printing and then heat transfer method can be applied to the large printing area and pad printing is good for small or curve area. The invention comprises composition of polymeric gel air freshener, printing ink composition, and methods of printing.

### Composition of Polymeric Gel Air Freshener

The products of this invention are useful for the air freshener and it is employed in the home (e.g. in room, closets, garages, storages, and cabinets), office, and vehicles. The gel air freshener of compositions comprises hydrocarbon oil, one or more triblock, radial block and/or multiblock copolymer and fragrances. The shape of gel air freshener can be mould in any shapes. Under this invention, varying the amount, ratio and types of polystyrene-rubber-polystyrene triblock copolymer controls the gel consistency of the air freshener. During the summer, the temperature inside the vehicle is around 60 to 65° C. To prevent the cracking of polymeric gel from the loop at high temperature inside the vehicles, the strength of gel air freshener must have higher softening temperature. The polymeric gel can retain its gel-like features over time; it is flexible, stretchable, and removable. In satisfaction of the foregoing advantages, the invention may accordingly be described as high softening gel air freshener comprising a styrene-rubber block polymer and no more than 300 parts by weight of hydrocarbon oil per 100 parts by weight of block copolymer. According to the present invention, gels are made of styrene rubber copolymer having weight average molecular weight Mw of at least 90,000, preferably at least 200,000, more preferably at least 300,000, and polystyrene content of 25 to 45 weight percent, preferably 28 to 40 weight percent, more preferably 30 to 35 weight percent. In general, the higher copolymer amount the stiffer the gel. Additionally, the higher amount of triblock, radial block and/or multiblock copolymer in polymer blend, the stiffer the blend gels. The gels under the present invention are generally transparent.

The composition of present polymeric gel air freshener blends a mixture of polymers in combination with hydrocarbon oil. The hydrocarbon oil used is desirably a natural or synthetic hydrocarbon oil of carbon chain length from 10 to 50. The oil may, for example, be a paraffinic oil, naphthenic oil or a natural mineral oil. The hydrocarbon oil can, for example, be natural or synthetic cosmetic grade hydrocarbon oil. Preferred the hydrocarbon oils are selected from paraffinic oil, naphthenic oil or natural oils, more preferably a white oil.

Commercially available thermoplastic rubber type copolymers, which are especially useful in forming the gel composition of the present invention, are sold under the trademark Septon and manufactured by Septon Company of America. They are available as a hydrogenated poly-isoprene/butadiene(SEEPS) polymer. The grade of the polymer is designated as Septon 4030, 4033, 4044, 4055, and 4077. Each molecule of SEEPS polymer consists of block segments of styrene monomer units and hydrogenated conjugated diene monomer units. The polystyrene block acts as a cross linking point at a temperature below the glass transition temperature of polystyrene. The rubber block acts as an origin of rubber-like properties; hydrogenation thereof provides excellent heat resistance and weather ability.

Suitable fragrances are generally known in the art. A wide variety of scents may be incorporated into the gel body including not only the conventional scents such as vanilla, citronella, burberry, floral, pine and the like, but also terpenes and essential oils. The combined or compounded fragrance should have a flash point of greater than 80° C., preferably at or above 95° C. The fragrance normally comprises a carrier solvent, such as diethylphthalate, carbitol, dipropyleneglycol, or dipropylglycol. Preferably 0.1 to 30 weight percent. Low molecular weight polyalphaolefin was added to keep rigidity of the gel air freshener after the addition of fragrances. The most unique characteristics of this highly branched alphaolefin polymers is the ability to bind oil, increase the hardness, very flexible and provide excellent lubricity properties. Commercially available polyalphaolefin polymers, which are especially useful in retaining the rigidity of gel composition of the present invention, are sold under the trademark VYBAR and manufactured by Baker Hughes Inc.

It is also advantageous to incorporate luminescent, fluorescent, pearlescent particles, glitters, metallic pigments, and optical brightener additives to gel air freshener, to add a degree of fun and extended function to the products. These types of additives can be applied to the thermoplastic tuber gel in accordance with manufacturer's guidelines. If desired, thermochromic pigments may be added to the gel air freshener. These pigments change color at predetermined temperatures. Alternatively the change may be from colored to colorless. Such thermochromic pigments are known per se and are commercially available from Matsui Inc. or The Pilot Ink Company of Japan. Any dye used should be oil soluble. Examples of suitable dyes are Blue 2B, Green GSB, Orange 3G, Red 2G, and Yellow 3G, sold under the name of Sandoplast by Clariant.

Other useful additives are light absorber to improve shelf stability of air freshener color when exposed to visible or ultraviolet light. The preferred light absorber is 2-)2-hydroxy-5-tert-cotylphenyl)benzotriazole, sold under the name Cyasorb UV-5411 light absorber by Cytec. With respect to antioxidants, The preferred product is bis(3,5-di-t-butyl-4-hydroxyhydrocinnamate), sold under the name of the Iragnox 1010 by Ciba Specialty chemicals. The additive amount is about from 0.05 to 0.1 weight percent.

A composition of formulation in accordance with the invention may be prepared by dispensing or injecting TPR gel into a shaped mould. The gel was allowed to set inside the mould at room temperature; and no further handling of the product is necessary. Preferably the air freshener is formed into an aesthetically eye catching shape.

The further improved device of the invention would allow an even more viable and flexible method of achieving a wide range of printed design. The addition of images could be potentially extremely beneficial to the marketing of the gel air freshener, as endorsed ‘in-car’ personal products are especially successful in today's current climate.

### Composition of Printing Inks

The present invention of printing ink applied on the surface of polymeric gel, includes resins, mineral oil, solvents, pigments or dyes, and additives. The process involves optionally preparing the basecoat treatment on the substrate of polymeric gel, printing multi-color image on the treated substrate of polymeric gel. The printing methods include pad printing, or screen printing or, heat transfer printing. A clear topcoat can be applied either by pad printing or spray method.

### Resins

The printing ink is composed of one or more resins. In most case the resins are obtained in the form of granulates or powders. The resins must be dissolved in a suitable solvent or solvents mixture. As a main component of the invention, the resins are responsible for the formation of the finished ink film and the carrier for the coloring material used in the ink formulation. The selection and combination of the resins determine the utilization of the ink's area and the resulting properties; such as adhesion to various substrates, grades of gloss, and resistance. Under the present invention, varying the amount and types of polymers affects the features of pad printing ink. For example, preferably using triblock, radical block and/or multiblock copolymers, and optionally a diblock copolymer. The printing ink, which has desirable rheological properties, will produce a durable and stretchable ink film. The polymers used comprise at least one copolymer selected from the radical block and/or multiblock copolymers. This invention contains at least two thermodynamically incompatible segments, one hard and one soft. In general, in a triblock polymer, the ratio of the segments is one hard, one soft, and one hard or an A-B-A copolymer. The multiblock and radical block copolymer can contain any combination of hard and soft segments. In the optional diblock copolymer, the blocks are sequential with respect to hard and soft segments.

Commercially available thermoplastic rubber type polymers are especially useful in forming the compositions of the present invention. Both Kraton Chemical Company and Septon Company of America sell commonly used polymers”. The most common structure is the linear ABA block type; styrene-butadiene-styrene (SBS) and styrene-isoprene-styrene (SIS) which is the Kraton D rubber series. Kraton G is another type of polymer. The copolymer comprises a styrene-ethylene-butylene-styrene (S-EB-S) structure. The Kraton G series is preferred in the practice of the invention. The optionally blended diblock polymers include the AB type such as styrene-ethylene-propylene (S-EP) and styrene-ethylene-butylene (S-EB), styrene-butadiene (SB) and styrene-isoprene (SI). Septon resins are available in either diblock (A-B) or the more common triblock) A-B-A) types. These include a hydrogenated poly-isoprene (S-EP, S-EP-S), a hydrogenated poly-isoprene/butadiene (S-EEP-S) polymer or a hydrogenated poly-butadiene (SEBS) polymer. Depending on the hardness of the substrate of the thermoplastic rubber compositions of ink, employing various combinations of triblock and radical block is necessary.

The printing ink preferably includes resins from about 1 to 12% by weight, more preferably from about 5 to 10% by weight, and still preferably from about 6 to 8% by weight.

### Mineral Oil

Mineral oils are highly refined, colorless, and odorless petroleum oil. A preferred mineral oil to mix with thermoplastic rubber of the invention is the so-called “white” mineral oil. This type of mineral oil is generally recognized as safe for contact with human skin. Mineral oil may be characterized in terms of its density and viscosity, where light mineral oil is relatively less viscous than heavy mineral oil.

Light mineral oils are preferred for use in the invention. Mineral oils are available commercially in both USP and NF grades. Examples of commercially available suitable mineral oils include Sonneborn® and Carnation® white oils from Witco, Isopar® K and Isopar® H from ExxonMobil, and Drakeol®, Draketex®, Parole white mineral oils from Penreco Company. The amount of mineral oil in the printing ink should range from about 10 to 30% by weight based on the total weight of pad printing ink components, preferably from about 15 to 25% by weight.

### Solvents

Solvents differ in their evaporation speeds and strengths. The amount of solvent in a printing ink is a major factor for its drying rate, printing speed and adhesion to the substrate. Solvents can be divided into thinners and retarders. Retarders are necessary when printing speed is slow and when drying ink system is extremely fast. Functioning as diluents in the corresponding ink system, thinners are a mixture of solvents. Mixing ink with thinners in the correct ratio to achieve the desired viscosity is extremely important. The viscosity of the final mixture will determine the effectiveness of the ink transfer. The type and amount of solvents will depend on the resins and pigment used in the ink system. In some cases, the substrates also play a role in determining which solvent should be used. The physical evaporation process of the solvents ink induces the drying of ink on substrate. At the same time the substrate of thermoplastic rubber compound is partially dissolved, the slight dissolution of the printing surface results in a direct bond between the ink and the substrate. In the present invention, top coat uses aromatic solvents to increase the adhesion between the ink film and the substrate of thermoplastic rubber and very low evaporation rate of glycol ether acetate are used to reduce the volatile of aromatic solvents. The solvents uses in this invention can be arranged in the following chemical group: aromatic hydrocarbon, ester, glycol ether acetate and ketone. For aromatic solvents, toluene, xylenes, aromatic 100, and aromatic 150 are preferred. From the ester group, isopropyl acetate and amyl acetate are preferred. In the glycol ether acetate group, propylene glycol methyl ether acetate, ethylene glycol monoethyl ether acetate and ethylene glycol monobutyl ether acetate are preferred. Lastly, cyclohexanone, diacetone alcohol, and isophorone are preferred from the Ketone group.

Preferred are compositions employing the combination of aromatic hydrocarbon, glycol ether acetate and ketone. The pad printing ink preferably includes solvents from about 30 to 80% by weight, more preferably from about 45 to 70% by weight.

### Colorants

Colorants provide the color tone of the ink and determine its hiding power. Colorants, either organic pigments or inorganic pigments, give color to a substrate by altering its reflective characteristics. There are hundreds of different types of pigment produced. Some are formed by nature in mineral or vegetable forms, but most are synthetic materials. When ink is applied to a substrate, colorants either remain on the surface or have a tendency to fill voids in irregular surfaces. The present invention contains a coloring agent that produces a desired color appearance. For this invention, organic pigments are preferred. The pigments may be those pigments suitable for use in printing ink; such type of pigment will be well known to those of ordinary skill in the art. Example of such pigments include, but are not limited to, pigment yellow 83 (C.I. 21108), pigment orange 34 (C.I. 21115), pigment red 48:3(C.I. 15865:3), pigment violet 23(C.I. 51319), pigment blue 15:2(C.I. 74160), pigment green 7 (C.I. 74260). Pigment white 6 (C.I. 77891) and pigment 7(C.I. 77266). In this invention, pigment makes up 10 to 30% by weight, preferably in an amount of about 15 to about 25% by weight.

### Additives

The additives are substances normally used in small quantities. Their function is to adjust the ink properties, such as flow, viscosity, or characteristic of the surface. Adhesion modifiers, matting powder, anti-foam agent, wetting agent, antioxidant, antistatic agents, and flow control agents are a few examples. However, solvents have the most profound effect on printing performance.

### Method of Printing

Normally in the method of pad printing, it lays down a very thin ink film ranging from 4 to 6 micron thick while the screen printing have much thicker ink on the substrate. The topcoat forms a stretch film sealing the printed image and preventing it from scratch. The compositions of topcoat are resins, mineral oil, additives and solvents. The selection of polymers in the clear coating is the same as those in pad printing ink. The content of resins in the clear coating ranges from approximately 5 to 30% by weight, more preferably from about 10 to 25% by weight, and ideally from about 15 to 20% by weight. It is necessary to use the retarder solvents for topcoat. Preferred are compositions employing the combination of aromatic solvents. The top coat should include solvent from about 35 to about 90% by weight, more preferably from about 50 to about 80% by weight, and ideally from about 60 to about 70% by weight. The solvent is used to make the irregular surface by dissolving the substrate of thermoplastic rubber. The mineral oil will be functioned as a retarder to prevent the either shrank or dissolution of the substrate due to the depth etching on the surface. The mineral oil is present in amounts ranging from about 5 to about 30% by weight, more preferably from about 10 to about 20% by weight.

### EXAMPLE 1

The multicolor pad-printing machine is used to print an image onto the polymeric gel, which contain up to 80% mineral oil. After having the image printing, the gel products containing fragrance are functioned as air freshener. Without fragrance added, the polymeric gel will become as a decorative ornament. The shapes of the gel can be smoothing convex such as ball, solid 3-D design features, and flat sheets. For example, in the three-color pad printing of a logo onto the surface of either ball products or flat sheets, the printing process is repeated four times, with three different colors of ink and one clear topcoat. The topcoat is applied by the pad printing method. Alternatively, the clear top coating can be applied by spray method. The method of the top coating should be selected based on the shape of the final products.

### EXAMPLE 2

The multicolor screen-printing machine is used to print an image onto the plastic release film. After having the image printing film, the automatic heat transfer machine is used to transfer the printing image to the gel products. The shapes of the gel products can be smoothing convex such as ball, and flat sheets. For example, in the three-color screen printing of a logo onto the surface of flat sheets, the printing process is repeated three times, with three different colors of ink and one clear topcoat. The topcoat is applied by the spray method. The method of the top coating should be selected based on the shape of the final products.

### EXAMPLE 3

Without the addition of fragrance, the printed gel products can be used as a removable window cling, or portable mobile phone holding aid and an anti-slip mat, universal in use with many other applications outside its primary intended use of helping to hold objects down and also when a sudden change motion may occur. It may also be used as a drink coaster

The invention has been described herein with the reference to certain preferred embodiments. It is understood that obvious variants thereon will become apparent to those skilled in the art. The invention is not to be considered as limited thereto.

REFERENCES TO BE CITED IN INFORMATION
DISCLOSURE STATEMENT

| Patent No. | Title of Invention |
|---|---|
| 1. U.S. Pat. No. 3,519,466 | Method For Printing |
| 2. U.S. Pat. No. 3,994,439 | Slow-Release Air Freshener Polymer-Blend |
| | Composition |
| 3. U.S. Pat. No. 4,369,284 | Thermoplastic Elastomer Gelatinous |
| | Composition |
| 4. U.S. Pat. No. 5,019,434 | Molded Slow-Release Air Freshener |
| 5. U.S. Pat. No. 5,618,882 | Gels Containing SEPS Block Polymers |
| 6. U.S. Pat. No. 5,871,765 | Non-Aqueous Controlled Release Pest and |
| | Air Care Gel Composition |
| 7. U.S. Pat. No. 5,884,639 | Crystal Gels With Improved Properties |
| 8. U.S. Pat. No. 6,168,088 | Air Freshener Card, Method of Use And |
| | Method of Manufacture |
| 9. U.S. Pat. No. 6,309,715 | Decorative Materials Encased in a Polymer |
| | With Fragrance Releasing Characteristics |
| 10. U.S. Pat. No. 6,357,260 | Scented Bracelet Kit |
| 11. U.S. Pat. No. 6,367,384 | Process For Applying 4 Color Image To A |
| | Fishing Lure |
| 12. U.S. Pat. No. 6,500,218 | Transparent Stiff Gel Candle |
| 13. US 20040169091 | Air Freshener and Method |

Foreign Patent Documents

| | |
|---|---|
| 1. GB 2 395 163 | A Plastic Mat with an Adhesive Surface and |
| | A method of Manufacture of Same |
| 2. GB 2 372 450 | Air Fresheners |

What is claimed:

1. A article of manufacture comprising decorative printing to polymeric gel air freshener comprising:
Composition of Polymeric gel air freshener;
Printing ink compositions; and
Printing methods.

2. A method according to claim 1 wherein the polymeric gel is thermoplastic rubber gel.

3. A method according to claim 1 wherein the polymeric gel comprise from about 5 to about 50% of a volatile hydrocarbon, one or more hydrocarbon-soluble substances, or mixtures hereof;
from about 10 to about 40% of a blend of triblock and/or multiblock copolymers, said blend comprising from about 100 to about 0 weight percent of one or more triblock, radial block and/or multiblock copolymer; and from about 0.1 to about 30 weight percent of fragrance.

4. The gel air freshener composition of claim 1 additionally comprising luminescent pigment, fluorescent pigment, thermochromic pigment metallic pigment, pearlescent particles and glitters.

5. The gel air freshener composition of claim 1 wherein said printing inks comprising colorants.

6. The gel air freshener composition of claim 1 additionally comprising one or more light absorbers.

7. The gel air freshener composition of claim 1 additionally comprising one or more antioxidants.

8. The gel air freshener of claim 1 wherein without addition of fragrance can be used as a window cling or anti-slip pad.

9. The printing ink composition of claim 1 further comprising:
(a) One or more copolymer resins selected from the group consisting of a triblock, radical block and multiblock copolymer, and optionally diblock copolymer:
(b) Mineral oil;
(c) Solvents;
(d) Colorants; and
(e) Additives.

10. The printing ink composition of claim 3, wherein each of said copolymers contains at least two thermodynamically incompatible segments, and are selected from the group consisting of:
(a) Styrene-butadiene-styrene polymers;
(b) Styrene-isoprene-styrene polymers;
(c) Styrene-ethylene-butadiene-styrene polymers;
(d) Styrene-ethylene-propylene-styrene polymers;
(e) Styrene-ethylene-butylene-styrene polymers;
(f) Styrene-butadiene polymers; and
(g) Styrene-isoprene polymers.

11. The printing ink composition of claim 4 wherein said copolymers comprise between about 1 to 12% by weight, more preferably from about 5% to 10% by weight.

12. The printing ink composition of claim 3 wherein said mineral oil comprise between from about 10 to 30% by weight, preferably from about 15% to 25% by weight.

13. The printing ink composition of claim 3 wherein said solvent comprises between from about 30% to 80% by weight, preferably from about 45% to 75% by weight.

14. The printing ink composition of claim 3 wherein said pigments comprise between from about 10% to 30% by weight, preferably from about 15% to 25% by weight.

15. The printing ink composition of claim 8 wherein said pigments further comprising:
luminescent pigment;
fluorescent pigment; and
thermochromic pigment.

16. The printing ink composition of claim 3 further comprising at least one additive selected from the group consisting of adhesion modifier, matting powder, antistatic agent, anti-foaming agent, wetting agent, antioxidant, and flow control agent, fragrance.

17. The top coat composition of claim 1 further comprising:
(a) One or more copolymer resins selected from the group consisting of a triblock, radical block and multiblock copolymer, and optionally diblock copolymer
(b) Mineral oil, other additive, and
(c) Solvents.

18. The top coat composition of claim 10, wherein each of said copolymers contains at least two thermodynamically incompatible segments, and are selected from the group consisting of:
(a) Styrene-butadiene-styrene polymers;
(b) Styrene-isoprene-styrene polymers;
(c) Styrene-ethylene-butadiene-styrene polymers;
(d) Styrene-ethylene-propylene-styrene polymers; and
(e) Styrene-ethylene-butylene-styrene polymers.

19. The top coat composition of claim 10 wherein said copolymers comprise between about 5 to about 30% by weight, more preferably from about 10 to about 25% by weight, and ideally about 15% to 20% by weight.

20. The top coat composition of claim 10 wherein said mineral oil comprises between from about 5% to 30% by weight, preferably from about 10% to about 20% by weight.

21. The top coat composition of claim 10 wherein said solvents comprise between from about 35% to 90% by weight, preferably from about 50% to about 80% by weight. More preferably from about 60% to about 70% by weight.

22. The top coat composition of claim 14 further comprising aromatic hydrocarbon, esters, glycol ether acetates and ketones

23. A printing method according to claim 1 wherein the printing ink is applied by either pad printing, screen printing, or spray coating onto the polymeric gel air freshener.

24. A printing method according to claim 1 wherein the printing ink is applied on release film by screen printing.

25. A printing method according to claim 24 wherein the ink printing release film is applied on polymeric gel air freshener by heat transfer machine.

26. A printing method according to claim 1 wherein the top coating is applied by pad printing onto the surface of printing image.

27. A printing method according to claim 24 wherein the top coating is applied by spray coating onto the surface of applied image.

28. An article of manufacture comprising a gel air freshener comprising:
(a) one or more copolymer resins selected from the group consisting of a triblock, radical block and muiltiblock copolymer, an optionally a diblock copolymer:
(b) mineral oil;
(c) solvent;
(d) additives; and
(e) a fragrance.
